(19) United States
(12) Patent Application Publication
(10) Pub. No.: US 2017/0245817 A1
(43) Pub. Date: Aug. 31, 2017

(54) SYSTEMS AND METHODS FOR IMAGING

(71) Applicants: Adel Malek, Boston, MA (US); THE CHARLES STARK DRAPER LABORATORY, INC., Cambridge, MA (US)

(72) Inventors: Andrew A. Berlin, Lexington, MA (US); Neil Gupta, Cambridge, MA (US); Rami S. Mangoubi, Newton, MA (US); Adel Malek, Boston, MA (US)

(21) Appl. No.: 15/429,993

(22) Filed: Feb. 10, 2017

Related U.S. Application Data
(60) Provisional application No. 62/293,431, filed on Feb. 10, 2016.

(51) Int. Cl. A61B 6/00; A61B 5/055; A61B 6/03; A61M 31/00

(52) U.S. Cl. CPC A61B 6/481; A61M 31/005; A61B 5/055; A61B 6/032; A61B 6/54

(57) ABSTRACT

A method of imaging an organism includes introducing a composite nanoparticle into a circulating fluid of an organism to form a circulating fluid mixture in the organism is provided. The composite nanoparticle comprises a core comprising at least one of a contrast agent and a magnetic material, and at least one layer of biocompatible material surrounding the core. The method further includes receiving an image of at least a portion of the organism where the circulating fluid has circulated, removing at least a portion of the circulating fluid mixture from the organism at a first rate, applying a magnetic field to the removed portion of the circulating fluid mixture to selectively remove the composite nanoparticle from the circulating fluid mixture and to produce a filtered fluid mixture, and returning the filtered fluid mixture to the circulating fluid of the organism at a second rate.

### CROSS-REFERENCE TO RELATED APPLICATIONS

This application claims priority under 35 U.S.C. §119(e) to U.S. Provisional Application Ser. No. 62/293,431 titled “REMOVABLE CONTRAST VASCULAR IMAGE ACQUISITION (ReCoVia),” filed Feb. 10, 2016, which is incorporated herein by reference in its entirety.

### FIELD OF TECHNOLOGY

One or more aspects of the disclosure relate generally to removable contrast agents for imaging, and to control systems for managing the level of a contrast agent in the body of an organism.

### SUMMARY

In accordance with one or more embodiments, systems and methods for imaging an organism are disclosed.

In accordance with one or more aspects, a method may comprise introducing a composite nanoparticle into a circulating fluid of an organism to form a circulating fluid mixture in the organism, the composite nanoparticle comprising a core comprising at least one of a contrast agent and a magnetic material, and at least one layer of biocompatible material surrounding the core. The method may further comprise receiving an image of at least a portion of the organism where the circulating fluid mixture has circulated, removing at least a portion of the fluid mixture from the organism at a first rate, applying a magnetic field to the removed portion of the circulating fluid mixture to selectively remove the composite nanoparticle from the circulating fluid mixture and to produce a filtered fluid mixture, and returning the filtered fluid mixture to the fluid of the organism at a second rate.

In some embodiments, the circulating fluid is blood.

In some embodiments, the circulating fluid is cerebrospinal fluid.

In some embodiments, the core of the composite nanoparticle is a magnetic material and the composite nanoparticle further comprises at least one layer of contrast agent in contact with the core and the at least one layer of biocompatible material.

In some embodiments, the method further comprises calculating at least one image analysis metric value of the image. In some embodiments, the image analysis metric value is an edge sharpness. In some embodiments, the image analysis metric value is a signal-to-noise ratio. In some embodiments, the method further comprises adjusting at least one of the first rate and the second rate based on the calculated image analysis metric value.

In accordance with one or more aspects, an imaging system may comprise a composite nanoparticle solution comprising composite nanoparticles, and an imaging device configured to display at least one image of a portion of an organism where the composite nanoparticle solution has circulated. The imaging system may further comprise a controller in communication with a source of the composite nanoparticle solution and the imaging device, and the controller configured to receive a first image from the imaging device, calculate at least one image analysis metric value from the first image, compare the calculated at least one image analysis metric value to a threshold value, and responsive to the comparison, adjust a rate of introduction of the composite nanoparticle solution to an organism.

In some embodiments, the at least one image analysis metric value includes at least one selected from the group consisting of: signal-to-noise ratio, edge sharpness, contrast, a resolution, artifacts, entropy, and distortion.

In some embodiments, the composite nanoparticle includes a core comprising at least one of a contrast agent and a magnetic material and at least one layer of biocompatible material surrounding the core. In some embodiments, the core of the composite nanoparticle is a magnetic material and the composite nanoparticle further comprises at least one layer of a contrast agent disposed between the core and the at least one layer of a biocompatible material.

In some embodiments, the controller is connected to at least one of a valve or a pump configured to introduce the composite nanoparticle solution to the organism.

In some embodiments, the controller is connected to at least one of a valve or a pump configured to withdraw a bodily fluid containing composite nanoparticles from the organism. In some embodiments, the at least one of a valve or a pump is fluidly connected to an inlet of the filtration device. In some embodiments, an outlet of the filtration device is fluidly connected to the organism. In some embodiments, the filtration device comprises at least one microfluidic device. In some embodiments, the filtration device is configured to filter the composite nanoparticles and produce a filtered bodily fluid. In some embodiments, the filtration device is configured to magnetically filter the composite nanoparticles.

In some embodiments, the imaging device is a magnetic resonance imaging device.

In some embodiments, the imaging device is an X-ray computed tomography device.

In some embodiments, the imaging device comprises a camera.

In some embodiments, the controller is coupled to a memory and is further configured to store the first image and the at least one image analysis metric value in the memory. In some embodiments, the controller is further configured to receive at least one second image from the imaging device, calculate at least one image analysis metric value from the second image, and compare the at least one calculated image analysis metric value from the second image to the at least one calculated image analysis metric value from the first image. In some embodiments, the controller is further configured to adjust the rate of introduction of the composite nanoparticle solution to the organism, responsive to the comparison of the at least one calculated image analysis metric values from the first and second images.

In some embodiments, the controller is further configured to notify a user when the calculated image analysis metric value deviates from the threshold value.

In some embodiments, responsive to the comparison, the controller is further configured to adjust a rate of withdrawal of a bodily fluid comprising the composite nanoparticles from the organism.

In accordance with one or more aspects, a method of facilitating an image of a portion of an organism comprises providing a composite nanoparticle and providing an instruction for introducing the composite nanoparticle into the organism, and providing an instruction for removing the composite nanoparticle from the organism.

In some embodiments, the method of facilitating further comprises providing an instruction for imaging a portion of the organism.

In some embodiments, providing an instruction for removing the composite nanoparticle from the organism further comprises instruction for filtering the composite nanoparticle to produce a filtered fluid, and returning the filtered fluid to the organism.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1A is a schematic of a cross-section of a composite nanoparticle in accordance with an embodiment;

FIG. 1B is a schematic of a cross-section of a composite nanoparticle in accordance with an embodiment;

FIG. 2 is a block diagram of an imaging system in accordance with an embodiment;

FIG. 3A is a schematic of one example of a component of a filtration device in accordance with an embodiment;

FIG. 3B is a schematic of another example of a component of a filtration device; and

FIG. 4 is a process flow chart of a method of imaging in accordance with an embodiment.

### DETAILED DESCRIPTION

Medical imaging is the technique and process of creating visual representations of the interior of a body for clinical analysis and medical intervention, as well as visual representation of the function of some organs or tissues. Some medical imaging methods, such as X-ray computed tomography (CT) scanning, magnetic resonance imaging (MRI), and ultrasound rely on the introduction of contrast agent into a patient's bloodstream. A contrast agent is a substance used to enhance the contrast of structures or fluids within the body. Contrast agents are commonly used to enhance the visibility of blood vessels and soft tissues within the body, and to trace the flow of blood through the vascular system of an organism.

Several types of contrast agents are used in medical imaging techniques. The contrast agents can be classified based on the imaging modalities for which they are used. For example, iodine and barium are the most common types of contrast agents for enhancing X-ray based imaging methods. The contrast agents may vary depending on their physical properties. For example, the contrast agents may vary based on their osmolarity, viscosity, and absolute iodine or barium content. In another example, gadolinium may be used as a contrast agent for MRI. The properties of the gadolinium contrast agent cause water around the agent to relax quickly, enhancing the quality of the MRI images.

Although the use of a contrast agent may be necessary to enhance medical imaging tests, the agents can sometimes lead to kidney problems, or may exacerbate the condition of patients with kidney disease. Two serious kidney disorders associated with contrast dyes are contrast induced nephropathy (CIN) and nephrogenic systemic fibrosis (NSF). Contrast agents may also be toxic to other bodily organs. The toxicity of the contrast agents may be affected by the rate of uptake of the contrast agent by the organ, the concentration of the contrast agent in the organ, and the length of time that the contrast agent interacts with the organ. Generally, the longer an amount of contrast agent interacts with an organ, such as the kidney or liver, the more likely it is to damage the organ. For this reason, a patient may not tolerate a large dose of a contrast agent, or an extended exposure to the contrast agent. Thus, the number of images that may be obtained for a patient may be limited.

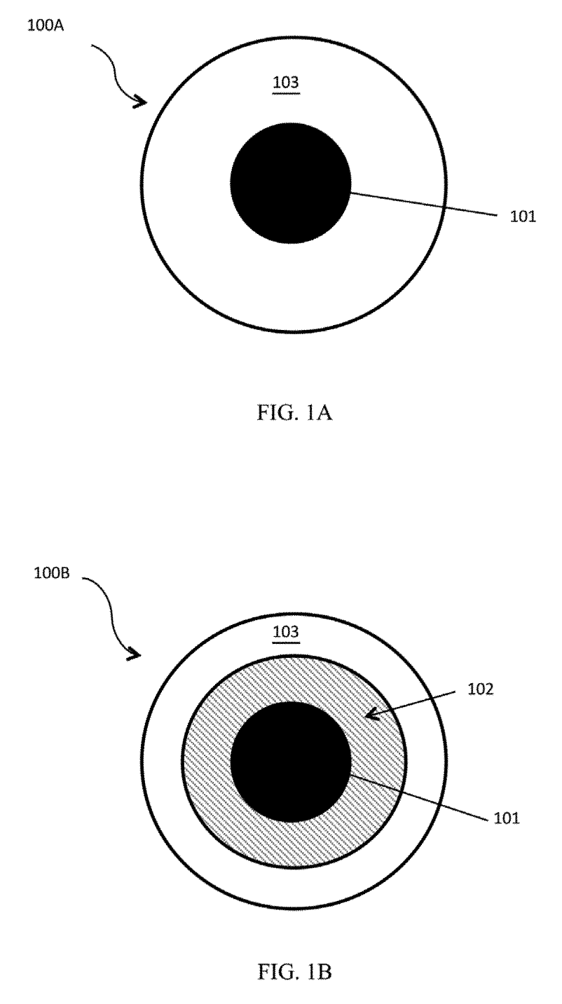

The disclosure is directed to systems and methods of managing the levels of a contrast agent in the body. Cross-sections of a composite nanoparticle 100 suitable for use in one or more embodiments is shown in FIGS. 1A and 1B as 100A and 100B respectively. Referring to FIG. 1A, the composite nanoparticle 100A may comprise a core 101 and a shell 103. In some embodiments, the core 101 may comprise at least one of a contrast agent and a magnetic material, and the core 101 may be encapsulated with at least one layer of a biocompatible material 103. The contrast agent of the core 101 may itself be magnetic, such as a contrast agent used in MRI such as Feridex or Resovist. In some embodiments, the core may be encapsulated by, or in contact with, one or more layers of biocompatible material. The biocompatible material may reduce the rate of uptake of the composite nanoparticle by bodily organs. As used herein, the term, “biocompatible,” when used in reference to a material, refers to a material having the ability to be in contact with the organism without producing an adverse effect. The biocompatible material may be inert with respect to the organism.

In accordance with some embodiments, and as shown in the composite nanoparticle 100B of FIG. 1B, the core 101 may be a magnetic material, and the composite nanoparticle may further include at least one layer of a contrast agent 102 in contact with the core and the at least one layer of biocompatible material 103. The contrast agent 102 may comprise one or more contrast medium materials that may be used to enhance the contrast of structures of fluids or tissue within the body of an organism during an imaging procedure. Non-limiting examples of contrast agents include x-ray blocking materials such as iodine or barium, and gadolinium which is often used in MRI imaging procedures. The layer of biocompatible material 103 functions to encapsulate the contrast agent in a biocompatible biologically inert material.

At least one physical property of the composite nanoparticle may facilitate preferential filtering from a bodily fluid of an organism. For example, at least one material forming at least one of the layers 101 and 102 of the composite nanoparticle may possess a physical property that allows it to be disambiguated or otherwise distinguished from other components of a bodily fluid. For instance, the core 101 of the composite nanoparticle may be magnetic, have an electrical charge pattern, and/or have a size, shape, mass, or density that permits separation forces to be preferentially applied to the composite nanoparticle relative to forces applied to other particles in the blood stream. In some embodiments, the composite nanoparticle comprises a magnetic core. For instance, the composite nanoparticle may comprise a ferrite core.

In some embodiments, the biocompatible shell layer 103 may be any biocompatible material, such as a biocompatible polymer. One example of a biocompatible polymer is polyethylene glycol. According to various embodiments, the biocompatible shell layer 103 may be any biocompatible co-polymer, such as poly(lactic-co-glycolic acid). In some embodiments, the biocompatible outer layer may be any biocompatible plastic. In accordance with one embodiment, the composite nanoparticle may comprise a ferrite core surrounded by a biocompatible polymer outer layer, such as polyethylene glycol. In this embodiment, the magnetism of the ferrite core provides a separation mechanism so that the composite nanoparticle can be removed from bodily fluid, and the radiopaque properties of the ferrite core allow it to be used as a contrast agent.

In accordance with some embodiments, the composite nanoparticle may have a diameter of from about 25 nm to about 1000 nm. According to one embodiment, the composite nanoparticle may have a diameter in a range of about 100 to about 200 nanometers, with 150 nm being a typical desirable dimension.

One or more methods may be used for producing the composite nanoparticles. Non-limiting examples of suitable methods include what is generally referred to as the “core-shell method” and the “one-pot method.” The core-shell method comprises forming a magnetic core from either a magnetic preformed material or a suitable metal-containing precursor material; coating the core with an inner layer comprising a contrast agent; and coating the inner layer with an outer layer comprising a biocompatible material. The one-pot method comprises providing a precursor mixture of a metallic material, and exposing the precursor mixture to the biocompatible material.

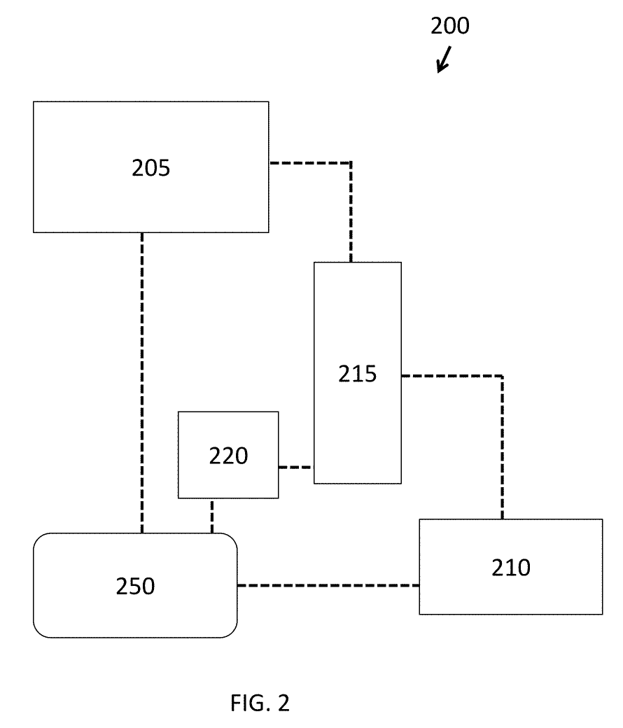

According to some embodiments, an imaging system, generally indicated at 200 in FIG. 2, may be used in combination with the composite nanoparticles discussed above for imaging an organism. The imaging system 200 comprises an imaging device 205, a filtration device 210, a composite nanoparticle solution 220, and a controller 250. One or more components of the imaging system 200 may be in communication with or otherwise coupled to one another, as indicated by the dashed lines in FIG. 2. As discussed in further detail below, the controller 250 may be coupled to or otherwise control at least one of the rate and the concentration at which the composite nanoparticle solution 220 is introduced to a bodily fluid of an organism 215, such as a human. Once the composite nanoparticle solution 220 is introduced to the organism, the imaging device 205 may obtain one or more images of the organism 215. The controller 250 may also be in communication with the imaging device 205, and may control one or more aspects of the imaging device. For instance, the controller 250 may send control signals to the imaging device 205 to power on/off, take an image, change image settings, etc. Although not explicitly shown in FIG. 2, a user may interface with the controller 250 and provide instructions to the controller to send control signals to the imaging device 205. When at least one image has been taken of the organism 215 by the imaging device 205, the filtration device 210 may function to remove the composite nanoparticles from the bodily fluid of the organism 215. The controller 250 may also control one or more operating parameters of the filtration device 210, such as powering on/off and the rate of removal of the composite nanoparticle. The controller 250 may also control the rate at which the filtered bodily fluid is re-introduced to the organism 215.

Although not explicitly shown in FIG. 2, the imaging system 200 may also include one or more pumps, syringes, valves, tubing, vessels, clamps, hypodermic needles, catheters, or other fluid flow control devices that may be used in combination with one or more of the controller 250, imaging device 205, filtering device 205, and composite nanoparticle solution 220.

According to some embodiments, use of the composite nanoparticles may include placing the composite nanoparticles in solution. For example, the composite nanoparticles may be suspended in a saline solution or in a dilute organic solvent. Colloidal chemistry encapsulants or reactants such as BSA (Bovine Serum Albumin) or salts may be employed to enhance or control the stability of the solution, controlling the rate of aggregation of the nanoparticles to form clusters. Depending on a particular application, the composite nanoparticles may be in a suspension or in a slurry. In some embodiments, the composite nanoparticles may be placed in a suspension or slurry and then introduced into a bodily fluid of an organism (e.g., a patient). In accordance with one embodiment, the composite nanoparticle solution 220 may be introduced to a bodily fluid of the organism 215. The bodily fluid may be a circulating fluid of the organism 215, such as blood or cerebrospinal fluid. The composite nanoparticles may be introduced to the circulating fluid of the organism 215 to form a circulating fluid mixture. According to some embodiments, the organism 215 is a human being, but in other embodiments, the organism 215 may be a non-human animal.

In one embodiment, the bodily fluid is a blood stream. According to other embodiments, the bodily fluid is cerebrospinal fluid. The composite nanoparticles may be introduced to the blood stream or cerebrospinal fluid at a controlled rate. The controller 250 may control and adjust the rate of introduction of the composite nanoparticles (e.g., the composite nanoparticle solution 220) to the organism 215. In some embodiments, controlling and adjusting the rate of introduction of the composite nanoparticles includes determining and controlling the rate of uptake by one or more of the bodily organs of the organism 215. For example, in the case of the use of X-ray CT imaging with digital-subtractive angiography to visualize blood flow within the brain (for instance to visualize aneurisms) a small bolus of contrast agent will be injected at a rate of 0.2 to 2.0 mL per second so as to provide a visualizable moving ‘edge’ of contrast agent that outlines the flow of blood within the brain. The rate of release of the contrast agent may be automatically adjusted by the controller 250 so as to maximize the contrast/sharpness of the moving ‘edge’ of contrast agent. In some embodiments, the rate of introduction of the composite nanoparticles may be controlled by a syringe connected to an IV catheter in a blood vessel of an organism. In other embodiments a fluidic dispenser such as an acoustically-driven pump, thermally-actuated ‘inkjet’ printhead, electrostatically-actuated dispenser, or other fluidic droplet dispensing mechanism may be utilized. The syringe may be controlled by the controller 250. In some embodiments, the rate of introduction of the composite nanoparticles may be controlled by the actuation of a valve connected to an intravenous (IV) catheter in a blood vessel of an organism. In some embodiments, the rate of introduction of the composite nanoparticles may be controlled by the operation of a pump controlled by the controller 250 and connected to an IV catheter in a blood vessel of an organism.

Upon introduction to the organism 215, the composite nanoparticle, e.g., composite nanoparticle solution 220, may mix with a bodily fluid in the organism 215 to form a fluid mixture. In some embodiments, the composite nanoparticle may mix with blood. In some embodiments, the composite nanoparticle may mix with cerebrospinal fluid. In some embodiments, the concentration of the nanoparticles in the bodily fluid may be about 5 mM to about 100 mM.

The fluid mixture may be removed from the organism 215 through, for example, an IV line that is in communication with the filtration device 210. According to at least one embodiment, the fluid mixture exiting the organism 215 may be directed to the filtration device 210, which may be configured to use a selective removal mechanism for filtering the composite nanoparticles out of the fluid mixture. The rate of removal of the fluid mixture and composite nanoparticle may be adjusted by the actuation of a valve or the operating of a pump in fluid communication with the organism 215 and the filtration device 210 and controlled by the controller 250, which may include a programmable logic controller (PLC). In some embodiments, a selective filtration mechanism may selectively remove the composite nanoparticles from the fluid mixture. Selectively filtering the composite nanoparticles, and therefore the contrast agent, further reduces the exposure of one or more bodily organs of the organism 215, such as the kidney, to the contrast agent. The selective filtration mechanism may include, for example, inline filtration of a patient's blood using a magnetic separation device. In some embodiments, the selective filtration mechanism may include, for example, filtration of a patient's blood that has been temporarily removed from a patient, and re-transfusing the filtered blood into the patient.

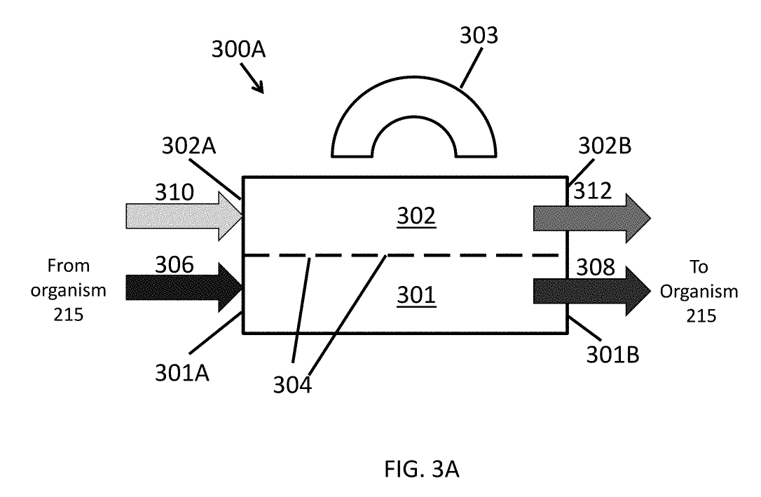

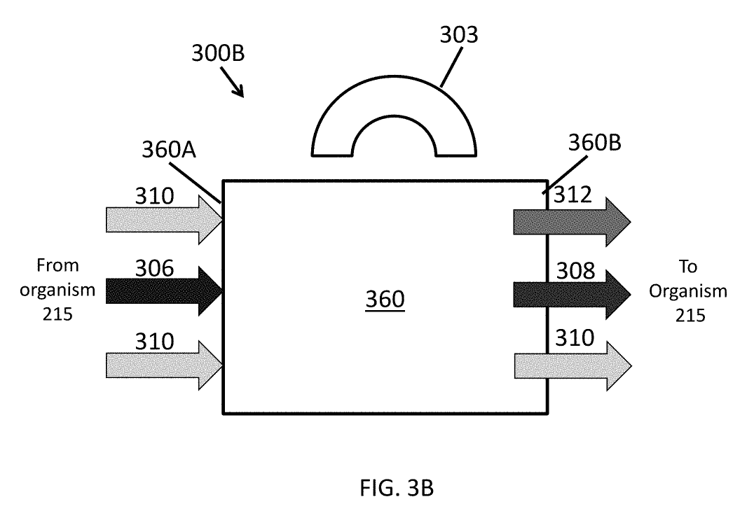

Referring to FIG. 3A, the filtration device 210 of FIG. 2 may comprise at least one microfluidic device 300 used for separating the composite nanoparticles from fluid mixtures exiting the organism 215. A cross-sectional view of one example of a microfluidic device 300A is shown in FIG. 3A. The microfluidic device 300A may include at least two adjacent fluidic channels 301, 302. The fluidic channels may be any shape, for example, rectangular or cylindrical. According to one embodiment, the diameter of the fluidic channels is less than 100 microns. In one embodiment, the dimensions of the channels will be chosen so as to achieve a laminar flow rather than turbulent flow. For example, the flow may have a Reynolds number of less than 100, or even less than 1. For comparison, turbulent flow typically occurs at Reynolds numbers in excess of 2000. For a small microfluidic channel, Reynolds number is calculated as:

$$Re = \frac{{LV}_{ρ}}{μ} \qquad ( 1 )$$

where Re is Reynolds number, L is the size scale (typically channel diameter), V is the average velocity of the fluid flow, ρ is the fluid density, and μ is the fluid viscosity.

According to the embodiment shown in FIG. 3A, the first channel 301 may be configured to accept an inflow of a bodily fluid mixture 306 from the organism at a first end 301A. The bodily fluid mixture 306 from the organism includes the composite nanoparticles that are used to produce images of the organism by the imaging device 205. The first channel 301 also includes a second end 301B that is configured to return an outflow of a filtered bodily fluid 308 back to the organism. The filtration device 210 may also include or be coupled to a source of buffer solution 310 that is used by the microfluidic device 300A for separating the composite nanoparticles form the bodily fluid mixture 306. The second channel 302 of the microfluidic device 300A may be configured to accept an inflow of the buffer solution 310 at a first end 302A. A second end 302B of the second channel 302 may be configured to remove a buffer solution mixture 312 comprising the buffer solution 308 and filtered composite nanoparticles as outflow. In some embodiments, the buffer solution may be a saline solution. According to certain embodiments, the buffer solution 310 may flow continuously at a predetermined flow rate. The predetermined flow rate may be a low flow rate, such as a flow rate in the Laminar flow regime. For instance, the buffer solution 310 may flow through fluidic channel 302 when a bodily fluid mixture 306 is introduced to fluidic channel 301. The flow rate of the buffer solution may be adjustable. According to some embodiments, the buffer solution 310 may flow in a predetermined time pattern. In some embodiments, the relative pressures of the buffer solution and the bodily fluid mixture may be adjustable.

The channels 301, 302 may comprise a series of open slits 304 that provide direct access and fluid communication between the first channel 301 and the second channel 302. The slits 304 may be of any shape and dimension suitable for allowing the composite nanoparticles to pass from the first channel 301 to the second channel 302. For example, the slits 304 may be rectangular or circular, and may be sized to be slightly larger than the diameters of the composite nanoparticles. The filtration device 210 may also comprise a mechanism for separating the composite nanoparticles from the bodily fluid mixture. The mechanism may be any mechanism that is capable of selectively separating the composite nanoparticles based on properties of the composite nanoparticles. For example, the filtration device 210 may comprise one or more magnets, such as the magnet 303 shown in FIG. 3A. Magnet 303 may be placed adjacent to the second channel 302. The magnet 303 may use magnetic forces as the separation mechanism to attract the magnetic composite nanoparticles from the first channel 301, and pull them up to the second channel 302 through the series of slits 304 extending through the first channel 301 to the second channel 302.

In accordance with some embodiments, the filtration device 210 may include multiple microfluidic devices such that they are configured as at least two first and second adjacent channel combinations. For example, a filtration device may comprise two or more sets of first and second adjacent channel combinations. The two sets of first and second adjacent channel combinations may be in a parallel configuration. In some embodiments, the first and second adjacent channel combinations may be in series configuration. For example, the filters may be in series configuration when the magnetic field strength or the channel dimension varies over the length of the multistage filter. In one embodiment, the magnetic field strength and/or the channel dimensions may vary such that the larger particles are removed from the bodily fluid mixture first, and the remaining particles are removed downstream. This may minimize the risk of particle aggregation and/or clogging of the system.

The filtration device 210 may comprise a plurality of microfluidic devices such as the microfluidic device 300A shown in FIG. 3A. According to some embodiments, the microfluidic device 300 may be a chip-based system and include a substrate, such as a silicon wafer or molded polymeric chip, that contain hundreds or thousands of the microfluidic devices arranged in an array to filter the bodily fluid mixture. In some embodiments, stacked silicon wafers may form a 3-dimensional filtration device. A three-dimensional filtration device may restrict the bodily fluid mixture from contacting a surface of the silicon wafer, thereby reducing the likelihood that some components of the bodily fluid may stick to the silicon and clot or clog the filtration device. For example, poly di-methyl silicon (PDMS) or stacked layers of polymethyl methacrylate (PMMA) may be used to form the multi-channel filtration device 210. Multiple magnets may also be used in the filtration device 210. The exact configuration of the filtration device 210 will depend on the application, including the bodily fluid being filtered, the physical composition of the organism being imaged, the various flow rates, the composition and size of the composite nanoparticles, and the type of imaging being performed.

A second example of a microfluidic device 300B is shown in FIG. 3B. In this instance, sample flow containing the bodily fluid mixture 306 is confined to the center of a microchannel 360 having a narrow-cross-section. In some embodiments, the diameter of the microchannel 360 is less than 100 microns. In some embodiments, the length of the microchannel 360 may be about 10 times to about 1000 times the diameter of the microchannel. The bodily fluid mixture 306 enters an inlet 360A of the microchannel 360 and exits an outlet 360B as filtered bodily fluid 308. Buffer solution 310 is introduced at the inlet 360A and exits the outlet 360B as the buffer solution mixture 312 that includes the filtered out composite nanoparticles 100. The magnet 303 provides a mechanism for the composite nanoparticles to transfer from the bodily fluid mixture 306 to the buffer solution 310 to generate buffer solution mixture 312. As shown in FIG. 3B, the buffer solution 310 functions as a carrier fluid for the composite nanoparticles to migrate from the bodily fluid mixture 306 to the buffer solution 310. The magnet 303 positioned adjacent a sidewall of the microchannel 360 allows the composite nanoparticles to move from the center of the flow channel and out of the bodily fluid toward the sidewall of the microchannel then exit the microchannel 360. The microchannel 360 may be a chip-based system where hundreds or thousands of microfluidic devices are prepared on a batch fabricated substrate and used by the filtration device 210.

Although the examples discussed herein include separation mechanisms based on magnetism, other separation mechanisms are also within the scope of this disclosure. For instance, the composite nanoparticles may be separated based on an electrical charge or electrical charge pattern, size, shape, mass, density, or any other property that allows for the preferential separation of the composite nanoparticle from the bodily fluid. Acoustic separation mechanisms such as acoustophoresis may also be used. According to another aspect, the separated composite nanoparticles may be recycled and reused. For instance, the composite nanoparticles may undergo a sterilization procedure and then be returned for use in another imaging procedure.

Referring back to FIG. 2, the imaging system 200 may also comprise an imaging device 205. In one embodiment, the imaging device is configured to display at least one image of the organism 215, and in some instances, displays at least one image of a portion of the organism 215. The portion of the organism that is displayed contains the nanocomposite particles and the contrast agent of the nanocomposite particles enhances the contrast of tissue or fluids within the organism. The amount of contrast agent in the image (and therefore the amount in the organism) influences the image quality. The composite nanoparticle solution 220 may be introduced to a circulating blood stream of the organism 215 and the imaging system 205 may obtain one or more images of a portion of the cardiovascular system of the organism 215. According to another embodiment, the composite nanoparticle solution 220 may be introduced to a circulating cerebrospinal fluid of the organism and the imaging system 205 may obtain one or more images of a portion of the ventricular system of the organism 215. Non-limiting examples of imaging devices include MRI, CT scanner, PET, and ultrasound devices, as well as fluoroscopes, nuclear scanners, and X-ray devices, and any other device configured to generate an anatomical image or representation of an organ or other tissue.

According to one embodiment, the imaging system 200 may comprise a control system. The control system may include the controller 250. In some embodiments, the introduction of the composite nanoparticles to the organism 215 may be controlled by the controller 250. The controller 250 may be in communication with a source of the composite nanoparticle solution. The controller 250 may provide an output signal to at least one of a pump or a valve of a syringe in communication with a source of composite nanoparticles, including the composite nanoparticle solution 220. The controller 250 may also provide an output signal to an outlet, such as a pump or valve in communication with a fluid mixture exiting the body of the organism 215 that contains the composite nanoparticles. The outlet may be in communication with the filtration device 210, which functions to remove the composite nanoparticles from the fluid mixture and return the filtered fluid to the organism 215. In some aspects, the image system may also include a timer that is part of the control system and that is in communication with the controller 250. As discussed in further detail below, in accordance with certain aspects the controller 250 is configured to calculate or otherwise determine at least one image analysis metric value from the image obtained by the imaging device 205. For example, the introduction and removal rates of the composite nanoparticles may be controlled by a process feedback mechanism based on one or more image analysis metric values. The control system, including the controller 250, may also include a memory that is communicatively coupled to the controller. In some instances, the memory is communicatively coupled to the controller through a communication network.

In some embodiments, the process feedback mechanism may be based on one or more qualitative parameters obtained from images taken of the organism's body by the imaging device 205. The imaging device 205 may be configured to obtain and display at least one image taken of the organism. The controller 250 may be configured to analyze the at least one image and in response, control a rate of introduction of the composite nanoparticles to the circulating bodily fluid of the organism 215. For instance, the controller 250 may increase or decrease the flow rate of the composite nanoparticle solution 220, or may increase or decrease a concentration of the composite nanoparticles in the composite nanoparticle solution 220. The controller 250 may also adjust one or more operating parameters of the imaging device 205 based on the analysis of the at least one image, such as power, exposure time, viewing angle, viewing distance, background settings, etc.

Images obtained by the imaging device 205 may be evaluated using image analysis metrics. In medical imaging, image quality may be determined by at least five image analysis metric values. Non-limiting examples include contrast, resolution, noise, artifacts, and distortion. Resolution, which may be described by a modulation transfer function, and noise, which can be measured by a noise power spectrum, are two of the most commonly used image analysis metric values. The modulation transfer function describes the ability of an imaging system to reproduce the frequency information contained in an incident X-ray signal. The noise power spectrum describes the frequency content of the noise of an imaging system. Image analysis metric values may vary amongst imaging systems and may therefore be dependent on the type of application being used. Another non-limiting example of an image analysis metric value is entropy. In some embodiments, information entropy may be the single parameter used to evaluate an image. Information entropy describes or otherwise captures how much randomness there is in a signal or an image. Other non-limiting examples of image analysis metrics include the contrast to noise ratio, the detective quantum efficiency, the image edge sharpness value, the signal-to-noise ratio, the local edge gradient value, and the wavefront of the contrast agent. A structural similarity index measure may also be used as the image quality metric. The structural similarity index functions by measuring the structural similarity that compares local patterns of intensities that have been normalized for luminance and contrast.

In accordance with certain embodiments, adjustment of the introduction and removal of the composite nanoparticles from the bodily fluid of the organisms may be based feedback regarding one or more image analysis metric values. In some instances, feedback may be based on automatic analysis, such as a value associated with image edge sharpness, user-provided analysis, or may be based on a predefined timing process, such as activating the filtering device 210 after a predetermined number of images have been obtained.

According to embodiments that encompass automatic feedback, the process feedback mechanism may be based on one or more image analysis metric values, including those mentioned above that are calculated by a computing device, such as an image processor (discussed further below). In addition, the time that it takes a contrast dye to move from a first predetermined location to a second predetermined location may be output as feedback.

According to embodiments that encompass user-provided feedback, the user may make an assessment of the image instead of a computing device. For instance, user-provided feedback may include an operator clicking on an icon indicating that the background is too dark. In other examples, a user may provide feedback that a vein bulges, for example, or that it leaks.

According to embodiments that encompass a predefined protocol, such as a predefined timing process, the feedback mechanism may be implemented by a controller, which may be a computer system. For instance, a controller may send an activation signal to the filtration device after five images have been acquired.

As noted above, adjustment of the introduction and removal of the composite nanoparticles from bodily fluid may be based on image parameter measurements, calculations, or algorithms performed on one or more images obtained by the imaging device 205. For example, the feedback may be based on the image sharpness of 2-dimensional to 3-dimensional image reconstruction techniques. The feedback input signal from the imaging device 205 may be converted to a controller 250 output signal that actuates a valve and/or pump to adjust the rate of introduction of the composite nanoparticles from a source of composite nanoparticles to the organism 215, or to adjust the rate of removal of the bodily fluid mixture from the organism 215. In some embodiments, the composite nanoparticles may be introduced to the bodily fluid from a source of composite nanoparticles through a syringe automatically as needed.

In accordance with various embodiments, the image system may include an image processor. The image processor may be a component of at least one of the imaging device 205 and the controller 250. The image processor may be configured to calculated and compare the image analysis metric value to a setpoint or threshold image analysis metric value or to a range of setpoint or threshold values. Responsive to the comparison, a rate of introduction of the composite nanoparticle solution to the organism may be adjusted. If the image analysis metric value deviates from the threshold or setpoint value, a valve in the syringe may open or close to facilitate or prevent introduction of composite nanoparticles to the circulating fluid of the organism, and a valve in the outlet may open or close, to facilitate or prevent removal of the circulating fluid comprising composite nanoparticles from the body. For example, the image processor may analyze the image and determine that the contrast to the surrounding tissue is too low. The image processor may send a signal to the controller 250, and in response, the controller may increase the rate of introduction of the composite nanoparticles to the organism 215. If the image analysis metric value is within the threshold value of the image analysis metric value, the system may maintain its configuration and operating parameters.

For imaging of the same area of the body, the imaging system may compare the average contrast, minimum brightness, and/or maximum brightness at the beginning of the exam and later in the exam. As more composite nanoparticles are introduced, the overall brightness will decrease over time, assuming that contrast appears as dark. When the image is too dark, the composite nanoparticles may be filtered from the body. In some embodiments, about a 20% or greater decrease in brightness of the image may require filtering of the composite nanoparticle from the body.

In some embodiments, a prior image may be used as a comparison point relative to the images received from the imaging device after the introduction of the composite nanoparticles. For example, the system may digitally subtract one image from the other. In this example, only the changes should appear. The changes are due to the newly introduced composite nanoparticles. The changes will appear with a particular intensity value relative to the background noise. In some embodiments, the intensity value may be greater than 5 signal-to-noise ratio. When the signal-to-noise ratio begins to fall, the composite nanoparticles may be filtered from the bodily fluid mixture.

In some embodiments, a pre-examination multi-dimensional set of images may be received. For example, a full 3-dimensional CT scan may be received prior to an operation. Projective imaging of one or two image planes may be performed during the procedure. The pre-operative scan may then map the projected image features into a 3-dimensional image. As the mapping algorithm, which may employ typical techniques such as expectation maximization, begins to have more uncertainty, the composite nanoparticles may be filtered from the bodily fluid mixture.

Although the examples discussed herein refer to instances where an image processor analyzes the image obtained from the imaging device, other examples where a user interfaces with the controller are also within the scope of this disclosure. For instance, a user may determine that the image does not have enough contrast, and may provide feedback to the controller to introduce more composite nanoparticles to the circulating fluid of the organism. Likewise, a user may determine that the quality of the obtained image(s) is sufficient for a particular application, including diagnostic functionality, and may therefore provide feedback to the controller that the rate of introduction of the composite nanoparticles may cease and/or the circulating bodily fluid may be allowed to exit the organism and be filtered.

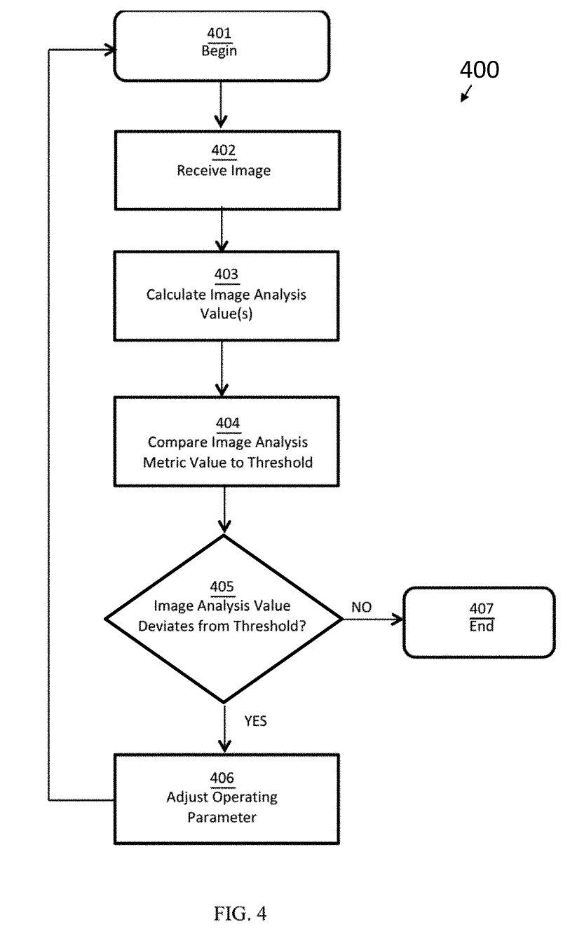

Referring now to FIG. 4, an example flow chart of a method 400 according to the processes disclosed herein is shown and begins at 401. According to some embodiments, the controller may be configured to receive images of an organism from an imaging device, such as an MRI or X-ray CT machine (step 402). As discussed above, the controller may comprise an image processor and therefore calculate at least one image analysis metric value (step 403) of the received image. After the image is analyzed, the controller may compare the calculated image analysis metric value to a baseline or a threshold image analysis metric value for images produced by the medical imaging device (step 404). Responsive to the comparison, the controller may adjust one or more operating parameters of the imaging system. For example, if the calculated image analysis value deviates from the threshold image analysis value, an operating parameter, such as a flow rate of a nanocomposite solution entering the organism or a flow rate of a fluid mixture entering the filtration device may be adjusted (step 406) by the controller. In some embodiments, the controller may adjust a concentration of composite nanoparticles that are introduced to a circulating bodily fluid of the organism. In some embodiments, the controller may adjust the rate of removal of circulating bodily fluid containing composite nanoparticles from the organism. For example, the signal-to-noise ratio value of the image may be compared to a threshold signal-to-noise ratio value by the controller. If the signal-to-noise ratio value falls below the threshold value, then the controller may adjust the concentration or flow rate of the composite nanoparticle solution entering the bodily fluid of the organism, and the process returns to step 401. If the calculated image analysis value does not deviate from the threshold image analysis value, operating parameters of the system may be maintained and the process ends (407). In some embodiments, the image analysis performed by controller 250 may include input from a human user, such as a subjective image quality score, indicating whether the imaging results being produced are meeting the needs of the practitioner.

The image obtained by the imaging device may include any image analysis metric value capable of being measured or calculated. For example, the image may include a signal-to-noise ratio value, an entropy value, and/or an edge sharpness or edge-definition value. The imaging device functions to capture one or more image(s) having measurable or calculable image analysis metric values and to submit the image(s) to the image processor for analysis, which in some instances is a component of the controller, for analysis. The imaging device can be operated by a user in some examples, while in others the imaging device operates automatically and is controlled by the controller. In some embodiments, an algorithm is implemented to train the processor to fine-tune the image analysis metric value threshold. The threshold criterion may also be determined in part by querying a database of typical threshold values that is indexed based upon the body part being imaged, the imaging modality being employed, the type of contrast agent being employed, the amount of contrast agent dispensed during the procedure, the patient's medical history (e.g., how much contrast agent have they been exposed to over their lifetime), and so forth. This indexed database may be updated based on the results of each procedure in which it is employed.

In accordance with certain embodiments, the methods and systems disclosed herein may be used to retrofit with a pre-existing imaging system. For instance, the controller 250, composite nanoparticle solution 220, and filtration device 210 may be combined as a “kit” that may be used in combination with a pre-existing imaging device 205.

According to certain aspects, a method of facilitating may be provided. The method may provide facilitating at least one of an imaging system, imaging device, and imaging of an organism. The method may provide facilitating one or more parts of a pre-existing imaging system. In accordance with one embodiment, the method of facilitating comprises providing a composite nanoparticle, such as the composite nanoparticle 100A and 100B shown in FIGS. 1A and 1B. The method may also comprise providing instructions for introducing the composite nanoparticle into the organism. The method may also comprise providing instructions for removing the composite nanoparticle from the organism. In some embodiments, the method may further comprise providing an instruction for imaging a portion of the organism.

In some embodiments, the method may also comprise introducing the composite nanoparticle into a circulating bodily fluid of an organism, as described above. The method may also comprise controlling the rate of introduction of the composite nanoparticle into the circulating bodily fluid of the organism. For instance, the flow rate of the composite nanoparticle solution 220 or the concentration of the composite nanoparticles in the solution 220 may be increased or decreased based on an assessment of an image analysis metric value calculated from an image received by the controller 250 of the organism. The method of facilitating may comprise providing at least one of a controller and filtration device as described herein.

It is to be appreciated that various alterations, modifications, and improvements will readily occur to those skilled in the art. Such alterations, modifications, and improvements are intended to be part of this disclosure, and are intended to be within the scope of the disclosure. For example, an existing system or process may be modified to utilize or incorporate any one or more aspects of the disclosure. Thus, in some cases, the systems and methods may involve connecting or configuring an existing system to comprise one or more of the composite nanoparticles, controller, and filtration device, and methods directed toward the same. Accordingly, the foregoing description and figures are by way of example only. Further, the depictions in the figures do not limit the disclosure to the characteristics of the particularly illustrated representations.

Various aspects and functions described herein may be included as specialized hardware or software components executing in one or more computer systems. One or more acts of the method described above may be performed with a computer, where at least one act is performed in a software program housed in a computer. Non-limiting examples of computer systems include, among others, network appliances, personal computers, workstations, mainframes, networked clients, servers, media servers, application servers, database servers and web servers. Other examples of computer systems may include mobile computing devices, such as cellular phones and personal digital assistants, and network equipment, such as load balancers, routers and switches. Further, aspects may be located on a single computer system or may be distributed among a plurality of computer systems connected to one or more communications networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, examples are not limited to executing on any particular system or group of systems. Further, aspects and functions may be implemented in software, hardware or firmware, or any combination thereof. Thus, aspects and functions may be implemented within methods, acts, systems, system elements and components using a variety of hardware and software configurations, and examples are not limited to any particular distributed architecture, network, or communication protocol.

A computer system may include a processor, a memory, an interconnection element, an interface and data storage element. To implement at least some of the aspects, functions and processes disclosed herein, the processor performs a series of instructions that result in manipulated data. The processor may be any type of processor, multiprocessor or controller. Some example processors include commercially available processors such as an Intel Atom, Itanium, Core, Celeron, or Pentium processor, an AMD Opteron processor, an Apple A4 or A5 processor, a Sun UltraSPARC or IBM Power5+ processor and an IBM mainframe chip. The processor may be connected to other system components, including one or more memory devices, by the interconnection element.

The memory may store programs and data during operation of the computer system. Thus, the memory may be a relatively high performance, volatile, random access memory such as a dynamic random access memory (“DRAM”) or static memory (“SRAM”). However, the memory may include any device for storing data, such as a disk drive or other nonvolatile storage device. Various examples may organize the memory into particularized and, in some cases, unique structures to perform the functions disclosed herein. These data structures may be sized and organized to store values for particular data and types of data.

Components of the computer system are coupled by an interconnection element such as the interconnection element. The interconnection element may include one or more physical busses, for example, busses between components that are integrated within a same machine, but may include any communication coupling between system elements including specialized or standard computing bus technologies such as IDE, SCSI, PCI and InfiniBand. The interconnection element enables communications, such as data and instructions, to be exchanged between system components of the computer system.

The computer system also includes one or more interface devices such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system to exchange information and to communicate with external entities, such as users and other systems.

The data storage element includes a computer readable and writeable nonvolatile, or non-transitory, data storage medium in which instructions are stored that define a program or other object that is executed by the processor. The data storage element also may include information that is recorded, on or in, the medium, and that is processed by the processor during execution of the program. More specifically, the information may be stored in one or more data structures specifically configured to conserve storage space or increase data exchange performance. The instructions may be persistently stored as encoded signals, and the instructions may cause the processor to perform any of the functions described herein. The medium may, for example, be optical disk, magnetic disk or flash memory, among others. In operation, the processor or some other controller causes data to be read from the nonvolatile recording medium into another memory, such as the memory, that allows for faster access to the information by the processor than does the storage medium included in the data storage element. The memory may be located in the data storage element or in the memory, however, the processor manipulates the data within the memory, and then copies the data to the storage medium associated with the data storage element after processing is completed. A variety of components may manage data movement between the storage medium and other memory elements and examples are not limited to particular data management components. Further, examples are not limited to a particular memory system or data storage system.

In some embodiments, the computer system may include specially programmed, special-purpose hardware, such as an application-specific integrated circuit (“ASIC”) tailored to perform a particular operation disclosed herein. While another example may perform the same function using a grid of several general-purpose computing devices running MAC OS X with IBM PowerPC processors and several specialized computing devices running proprietary hardware and operating systems.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

The systems and methods disclosed herein may be used to enable contrast-enhanced imaging of any fluid-flow system, such as an aircraft hydraulic system or chemical factory/refinery infrastructure, in which introduction of a contrast agent, imaging, and selective filtration of contrast agent is employed. For example, the systems and methods disclosed herein may be used for leak and conduit defect detection, sizing, and localization.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A method comprising:
introducing a composite nanoparticle into a circulating fluid of an organism to form a circulating fluid mixture in the organism, the composite nanoparticle comprising:
a core comprising at least one of a contrast agent and a magnetic material; and
at least one layer of biocompatible material surrounding the core;
receiving an image of at least a portion of the organism where the circulating fluid mixture has circulated;
removing at least a portion of the circulating fluid mixture from the organism at a first rate;
applying a magnetic field to the removed portion of the circulating fluid mixture to selectively remove the composite nanoparticle from the circulating fluid mixture and to produce a filtered fluid mixture; and
returning the filtered fluid mixture to the circulating fluid of the organism at a second rate.

2. The method of claim 1, wherein the circulating fluid is blood.

3. The method of claim 1, wherein the circulating fluid is cerebrospinal fluid.

4. The method of claim 1, wherein the core of the composite nanoparticle is a magnetic material and the composite nanoparticle further comprises at least one layer of contrast agent in contact with the core and the at least one layer of biocompatible material.

5. The method of claim 1, further comprising calculating at least one image analysis metric value of the image.

6. The method of claim 5, wherein the at least one image analysis metric value is an edge sharpness.

7. The method of claim 5, wherein the at least one image analysis metric value is a signal-to-noise ratio.

8. The method of claim 5, further comprising adjusting at least one of the first rate and the second rate based on the calculated image analysis metric value.

9. An imaging system comprising:
a composite nanoparticle solution comprising composite nanoparticles;
an imaging device configured to display at least one image of a portion of an organism where the composite nanoparticle solution has circulated; and
a controller in communication with a source of the composite nanoparticle solution and the imaging device and configured to:
receive a first image from the imaging device,
calculate at least one image analysis metric value from the first image;
compare the calculated at least one image analysis metric value to a threshold value; and
responsive to the comparison, adjust a rate of introduction of the composite nanoparticle solution to the organism.

10. The imaging system of claim 9, wherein the at least one image analysis metric value includes at least one selected from the group consisting of: signal-to-noise ratio, edge sharpness, contrast, resolution, artifacts, entropy, and distortion.

11. The imaging system of claim 9, wherein the composite nanoparticle includes a core comprising at least one of a contrast agent and a magnetic material and at least one layer of biocompatible material surrounding the core.

12. The imaging system of claim 11, wherein the core of the composite nanoparticle is a magnetic material and the composite nanoparticle further comprises at least one layer of contrast agent disposed between the core and the at least one layer of biocompatible material.

13. The imaging system of claim 9, wherein the controller is connected to at least one of a valve or a pump configured to introduce the composite nanoparticle solution to the organism.

14. The imaging system of claim 9, wherein the controller is connected to at least one of a valve or a pump configured to withdraw a bodily fluid containing composite nanoparticles from the organism.

15. The imaging system of claim 14, wherein the at least one of a valve or pump is fluidly connected to an inlet of a filtration device.

16. The imaging system of claim 15, wherein an outlet of the filtration device is fluidly connected to the organism.

17. The imaging system of claim 5, wherein the filtration device comprises at least one microfluidic device.

18. The imaging system of claim 15, wherein the filtration device is configured to filter the composite nanoparticles and produce a filtered bodily fluid.

19. The method of claim 18, wherein the filtration device is configured to magnetically filter the composite nanoparticles.

20. The imaging system of claim 1, wherein the imaging device is a magnetic resonance imaging device.

21. The imaging system of claim 12, wherein the imaging device is an X-ray computed tomography device.

22. The imaging system of claim 9, wherein the imaging device comprises a camera.

23. The imaging system of claim 9, wherein the controller is coupled to a memory and is further configured to store the first image and the at least one image analysis metric value in the memory.

24. The imaging system of claim 23, wherein the controller is further configured to:
receive at least one second image from the imaging device;
calculate at least one image analysis metric value from the second image; and
compare the at least one calculated image analysis metric value from the second image to the at least one calculated image analysis metric value from the first image.

25. The imaging system of claim 24, wherein the controller is further configured to adjust the rate of introduction of the composite nanoparticle solution to the organism, responsive to the comparison of the at least one calculated image analysis metric values from the first and second images.

26. The imaging system of claim 9 wherein the controller is further configured to notify a user when the calculated image analysis metric value deviates from the threshold value.

27. The imaging system of claim 9, wherein responsive to the comparison, the controller is further configured to adjust a rate of withdrawal of a bodily fluid comprising the composite nanoparticles from the organism.

28. A method of facilitating an image of a portion of an organism, comprising:
providing a composite nanoparticle;
providing an instruction for introducing the composite nanoparticles into the organism; and
providing an instruction for removing the composite nanoparticle from the organism.

29. The method of facilitating of claim 28, further comprising providing an instruction for imaging a portion of the organism.

30. The method of facilitating of claim 28, wherein providing an instruction for removing the composite nanoparticle from the organism further comprises instruction for filtering the composite nanoparticle to produce a filtered fluid, and returning the filtered fluid to the organism.
